(19) United States
(12) Patent Application Publication
(10) Pub. No.: US 2017/0086739 A1
(43) Pub. Date: Mar. 30, 2017

(54) MEASURING APPARATUS AND MEASURING SYSTEM

(71) Applicant: KYOCERA Corporation, Kyoto (JP)

(72) Inventor: Yuji MASUDA, Yasu-shi (JP)

(73) Assignee: KYOCERA Corporation, Kyoto (JP)

(21) Appl. No.: 15/264,858

(22) Filed: Sep. 14, 2016

(30) Foreign Application Priority Data
Sep. 28, 2015 (JP) 2015-190427

(51) Int. Cl. A61B 5/00; A61B 5/024

(52) U.S. Cl. CPC A61B 5/681; A61B 5/024; A61B 5/7278; A61B 5/742

(57) ABSTRACT

A measuring apparatus includes a wearing portion to be worn by a subject, a sensor unit for acquiring the biological information of the subject while being supported by the wearing portion and in contact with a test site of the subject, and a protrusion disposed around the sensor unit. The protrusion protrudes further than the sensor unit toward the subject when the sensor unit is not in contact with the test site. When the wearing portion is worn by the subject, the protrusion contacts with a periphery of the test site and deforms before the sensor unit contacts with the test site, thereby stretching the skin of the test site to be contacted by the sensor unit.

### CROSS-REFERENCE TO RELATED APPLICATION

This application claims priority to and the benefit of Japanese Patent Application No. 2015-190427 file Sep. 28, 2015, the entire contents of which are incorporated herein by reference.

### TECHNICAL FIELD

This disclosure relates to a measuring apparatus for measuring biological information and a measuring system including such a measuring apparatus.

### BACKGROUND

Conventionally, there has been known a measuring apparatus for measuring biological information from a test site such as a subject's (user's) wrist and the like.

### SUMMARY

A measuring apparatus according to the disclosure herein includes:

a wearing portion to be worn by a subject,

a sensor unit for acquiring biological information of the subject while being supported by the wearing portion and in contact with a test site of the subject; and

a protrusion disposed around the sensor unit, wherein

the protrusion protrudes further than the sensor unit toward the test site when the sensor unit is not in contact with the test site, and

the protrusion, when the wearing portion is worn by the subject, contacts with a periphery of the test site and deforms before the sensor unit contacts with the test site, thereby stretching the skin of the test site to be contacted by the sensor unit.

A measuring apparatus according to the disclosure herein includes:

a sensor unit for acquiring biological information of a test site; and

a protrusion disposed around the sensor unit, wherein

the protrusion, when a portion thereof protruding from the sensor unit toward the test site contacts with a part other than the test site, stretches the skin of the test site, and

the sensor unit acquires the biological information while being in contact with the skin of the test site being stretched.

A measuring apparatus according to the disclosure herein includes:

a wearing portion to be worn by a subject;

a sensor unit for acquiring biological information of the subject while being supported by the wearing portion and in contact with a test site of the subject; and

a protrusion disposed around the sensor unit, wherein

the protrusion at least partially has a shape spreading toward the test site and, when the sensor unit is not in contact with the test site, protrudes further than the sensor unit toward the test site, and

the protrusion, when the sensor unit contacts with the test site, contacts with a periphery of the test site and deforms further spreading the shape.

### BRIEF DESCRIPTION OF THE DRAWINGS

In the accompanying drawings:

FIG. 1 is a perspective view illustrating appearance of a measuring apparatus according to an embodiment of the disclosure herein;

FIG. 2 is a schematic diagram illustrating a state in which the measuring apparatus according to the embodiment of the disclosure herein is worn by a subject;

FIGS. 3A and 3B are an elevation view and a side view, respectively, illustrating the appearance of the measuring apparatus according to the embodiment of the disclosure herein;

FIG. 4 is a lateral cross-sectional view illustrating a structure of the measuring apparatus according to the embodiment of the disclosure herein;

FIGS. 5A to 5C are lateral cross-sectional views illustrating a state in which the measuring apparatus according to the embodiment of the disclosure herein is pressed against a test site;

FIGS. 6A and 6B are elevation views illustrating an example of the appearance of the measuring apparatus according to the embodiment of the disclosure herein;

FIGS. 7A and 7B are elevation views illustrating still another example of the appearance of the measuring apparatus according to the embodiment of the disclosure herein;

FIG. 8 is a schematic diagram illustrating a state in which the measuring apparatus illustrated in FIG. 7 is worn by the subject;

FIGS. 9A and 9B are diagrams illustrating examples of a pulse wave acquired by the measuring apparatus illustrated in FIG. 7;

FIG. 10 is a functional block diagram illustrating a schematic configuration of the measuring apparatus illustrated in FIG. 7; and

FIG. 11 is a diagram schematically illustrating a measuring system which includes the measuring apparatus according to the embodiment of the disclosure herein.

### DETAILED DESCRIPTION

When measuring the biological information by using the measuring apparatus described in the PLT 1 or the PLT 2, depending on a condition at a position of the wrist where the subject is wearing the measuring apparatus by using a belt, noise may be increased in measurement. It could be helpful to provide a measuring apparatus and a measuring system those capable of improving accuracy in the measurement of the biological information.

Hereinafter, an embodiment of the disclosure herein will be described in detail with reference to the accompanying drawings.

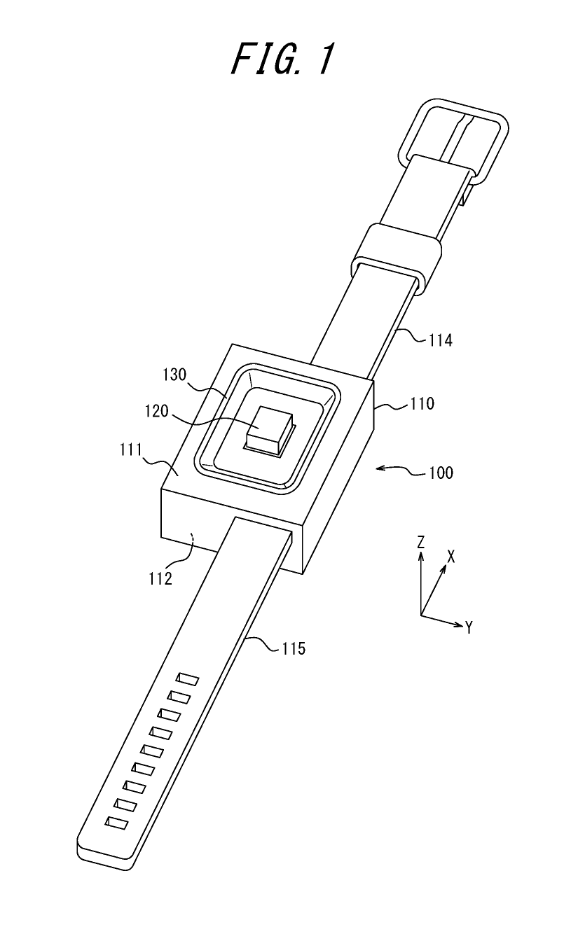

FIG. 1 is a perspective view illustrating a schematic structure of a measuring apparatus according to one embodiment of the disclosure herein. A measuring apparatus 100 according to the disclosure herein includes, in appearance, a wearing portion 110, a sensor unit 120, and a protrusion 130. As illustrated in FIG. 1, the wearing portion 110 of the measuring apparatus 100 includes a rear surface 111 facing a positive direction of a Z-axis illustrated in the figure and a front surface 112 facing a negative direction of the Z-axis.

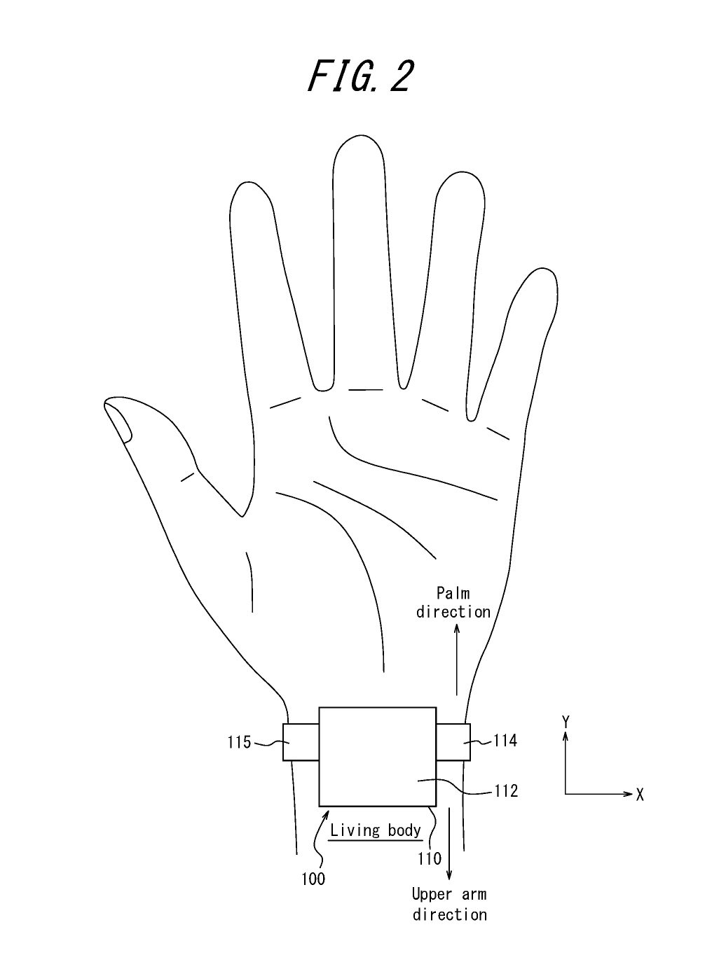

FIG. 2 is a schematic diagram illustrating a state in which the measuring apparatus 100 is worn by a subject. FIG. 2 is a diagram viewing the measuring apparatus 100 illustrated in FIG. 1 in the positive direction of the Z-axis (from a side of the front surface 112 of the wearing portion 110). The measuring apparatus 100 is used with the rear surface 111 of the wearing portion 110 being in contact with a test site of a subject's living body. As illustrated in FIG. 2, accordingly, in the state in which the subject is wearing the wearing portion 110 of the measuring apparatus 100, the subject may view the front surface 112 of the wearing portion 110. FIG. 2 illustrates, by way of example, a state in which the measuring apparatus 100 is used to measure biological information with the sensor unit 120 being in contact with the test site at the wrist on a palm side (of the left hand) of the subject.

As illustrated in FIG. 2, the wearing portion 110 is used when worn by the subject. Therefore, the wearing portion 110 may include a housing body (the wearing portion 110) illustrated in the figure and, also, for example, members such as belt portions 114 and 115. FIG. 1 and FIG. 2 illustrate, by way of example, the belt portions 114 and 115 which are used to wind around the arm or the like of the subject. Such belt portions 114 and 115 are not limited to designs thereof as illustrated in FIG. 1 and FIG. 2 but may have any design wearable by the subject. According to the disclosure herein, as described above, the wearing portion 110 may be a belt to be worn by the subject on the wrist.

As illustrated in FIG. 1, the wearing portion 110 of the measuring apparatus 100 has an opening in the vicinity of a center of the rear surface 111, and the sensor unit 120 protrudes from the opening in the positive direction of the Z-axis. The sensor unit 120 acquires the biological information of the test site. The sensor unit 120 acquires the biological information of the subject while being supported by the wearing portion 110 and in contact with the test site of the subject. The sensor unit 120, as described later, is supported by the wearing portion 110 via an elastic body. Therefore, when the wearing portion 110 of the measuring apparatus 100 is pressed against the test site of the subject, a protruding amount of the sensor unit 120 slightly decreases based on a pressure. Thereby, the sensor unit 120 contacts with the test site of the subject applying a fixed pressure thereto.

In the measuring apparatus 100 of the disclosure herein, the protrusion 130 is disposed around the sensor unit 120. As illustrated in FIG. 1, the protrusion 130 has a shape protruding toward the test site of the subject, i.e., protruding in the positive direction of the Z-axis and spreading.

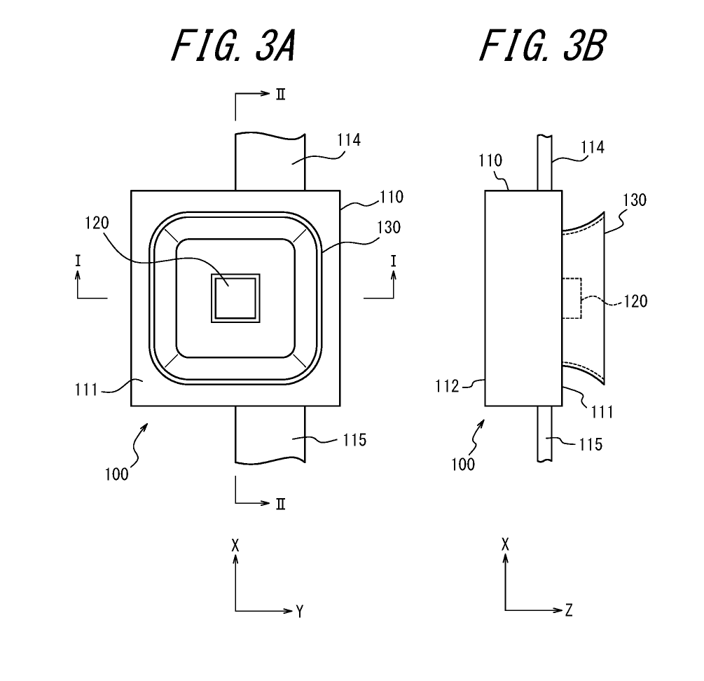

FIGS. 3A and 3B are an elevation view and a side view illustrating appearance of the measuring apparatus 100. FIG. 3A is an elevation view mainly illustrating appearance of the wearing portion 110 of the measuring apparatus 100. That is, FIG. 3A is a diagram viewing the measuring apparatus 100 illustrated in FIG. 1 in the negative direction of the Z-axis (from a side of the rear surface 111 of the wearing portion 110). FIG. 3B is a side view mainly illustrating the appearance of the wearing portion 110 of the measuring apparatus 100. That is, FIG. 3B is a diagram viewing the measuring apparatus 100 illustrated in FIG. 1 in a positive direction of a Y-axis. In FIG. 3A and FIG. 3B, the belt portions 114 and 115 are partially illustrated.

As illustrated in FIG. 3A and FIG. 3B, the wearing portion 110 of the measuring apparatus 100 has the opening in the vicinity of the center of the rear surface 111, and the sensor unit 120 protrudes from the opening in the positive direction of the Z-axis. Also, the protrusion 130 is disposed around the sensor unit 120 and protrudes in the positive direction of the Z-axis. Further, the protrusion 130 has a shape spreading toward the test site of the subject, i.e., in the positive direction of the Z-axis. Although in FIG. 3B the protrusion 130 has a shape spreading in a curving fashion in the positive direction of the Z-axis, the protrusion 130 is not limited to such a shape but may have a shape spreading, for example, in a linear fashion in the positive direction of the Z-axis.

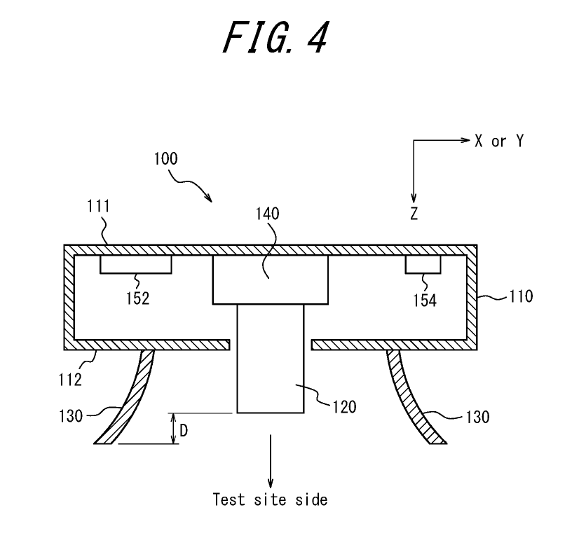

FIG. 4 is a cross-sectional view taken from line I-I or II-II of FIG. 3A. Hereinafter, the cross-section taken from the line I-I and the cross-section taken from the line II-II are correctively described on the assumption that these cross-sections have approximately the same structure. However, the disclosure herein is not limited to such a structure but the cross-sections may have structures different from each other. Note that in FIG. 4 the belt portions 114 and 115 are omitted.

As illustrated in FIG. 4, inside the wearing portion 110 of the measuring apparatus 100, the sensor unit 120 is supported by the wearing portion 110 via an elastic body 140. Also, the protrusion 130 is disposed around the sensor unit 120 and protrudes toward the test site of the subject (in the positive direction of the Z-axis). According to the disclosure herein, further, the protrusion 130, when the sensor unit 120 is not in contact with the test site of the subject, protrudes further than the sensor unit 120 toward the test site of the subject (by a distance D).

The following is a further description of each constituent of the measuring apparatus 100 with reference to FIG. 4.

The wearing portion 110 constitutes the housing body of the measuring apparatus 100 and, when including the belt portions 114 and 115, may constitute, as a whole, a belt to be worn by the subject on the wrist and the like. The wearing portion 110 constituting the housing body of the measuring apparatus 100 may be made by using a relatively robust material such as, for example, plastic, synthetic resin, and any metal. The wearing portion 110 may be produced by integrally molding in its entirety or by assembling any number of parts. Also, the wearing portion 110 is not limited to the shape illustrated in FIG. 1 to FIG. 4 but may have any shape depending on various usage, specifications, and the like.

On the other hand, since the belt portions 114 and 115 used together with the wearing portion 110 constituting the housing body of the measuring apparatus 100 are wound around the subject's wrist and the like, the belt portions 114 and 115 need to be able to bend in its entirety. The belt portions 114 and 115, as illustrated in FIG. 1, may have a design employed for a belt of a watch. However, the belt portions 114 and 115 are not limited to a design or a shape illustrated in FIG. 1 but may have various designs or shapes. The belt portions 114 and 115 may be removably worn by the subject at the test site on the wrist and the like and firmly fixed to the test site during measurement. The belt portions 114 and 115 may be made of various materials such as, for example, elastomer, synthetic fibers, natural leather, or artificial leather.

The sensor unit 120 may be constituted by using various sensors which, when in contact with the test site of the subject, acquires the biological information of the subject. For example, the sensor unit 120 may be constituted by using one or more light emitting elements for irradiating light to the test site. The sensor unit 120 may be constituted by using, for example, one or more photodetectors for detecting light irradiated from the light emitting element and travelled through the living body. Although FIGS. 1 to 4 illustrate the sensor unit 120 in the shape of a square column, the sensor unit 120 is not limited to such a shape but may be in any shape in accordance with various use and specifications. For example, the sensor unit 120 may be in a rectangular shape, a cylindrical shape, or an elliptic cylindrical shape, instead of a square column shape. A portion of the sensor unit 120 to come into contact with the test site may have a convex shape. When the portion of the sensor unit 120 to come into contact with the test site has the convex shape, the sensor unit 120 may maintain stable contact with the test site of the subject. The sensor unit 120 may be designed in various manners, and a specific example of the sensor unit 120 used for the measuring apparatus 100 according to the disclosure herein will be described in detail later.

The protrusion 130, as illustrated in FIG. 4, has a shape at least partially spreading toward the test site of the subject. As described above, when the sensor unit 120 is not in contact with the test site of the subject, the protrusion 130 protrudes further than the sensor unit 120 toward the test site. FIG. 4 illustrates a state in which the protrusion 130 protrudes further than the sensor unit 120 toward the test site by the distance D. As described later, when the protrusion 130 contacts with and is pressed against the skin of the test site, the shape of the protrusion 130 originally spreading toward the test site further spreads. Therefore, the protrusion 130 may be designed to at least partially have elasticity or flexibility. The protrusion 130 may be made by using a material having appropriate hardness so as to deform as being pressed against the skin when the measuring apparatus 100 is worn, having high impact resilience, unlikely to generate cracks due to repeating deformation, and having excellent water resistance. The protrusion 130 may be made of materials such as, for example, silicone rubber, natural rubber, nitrile rubber, fluoro rubber, ethylene vinyl acetate rubber, and the like.

As illustrated in FIG. 4, the elastic body 140 may lie between the wearing portion 110 and the sensor unit 120. The elastic body 140 has a function to apply elasticity to the sensor unit 120 and thus push the sensor unit 120 in a direction of the test site (in the positive direction of the Z-axis). The elastic body 140 may be made by using various elastic members such as, for example, urethane, rubber, a spring, and a leaf spring. In FIG. 4, the elastic body 140 uses repulsive force in accordance with pressing force as force used to restore a position of the sensor unit 120. However, the elastic body 140 is not limited to such a structure but may be constituted by using a spring having a function to pull the sensor unit 120 in a direction in which the sensor unit 120 protrudes from the opening.

Circuits 152 and 154 are various circuits necessary for the measuring apparatus 100 to measure the biological information by using the sensor unit 120. For example, the circuits 152 and 154 may be driving circuits of the sensor unit 120. The circuits 152 and 154 may be processing units for performing various information processing on the biological information acquired by the sensor unit 120. The circuits 152 and 154 may be memory for storing the biological information acquired by the sensor unit 120, a result of the processing on the biological information, and the like. Further, the circuits 152 and 154 may be other sensor units for acquiring information other than the biological information acquired by the sensor unit 120. On the other hand, when the measuring apparatus 100 is configured to output the biological information acquired by the sensor unit 120 as it stands to the outside, the circuits 152 and 154 may be omitted. In such a configuration, for example, a transmission unit for wirelessly output the biological information acquired to the outside, or an interface (a terminal or the like) for outputting, in a wired manner, the biological information acquired may be provided. In the following description, the circuits 152 and 154 are omitted.

Next, the measurement of the biological information by using the measuring apparatus 100 according to the disclosure herein will be described.

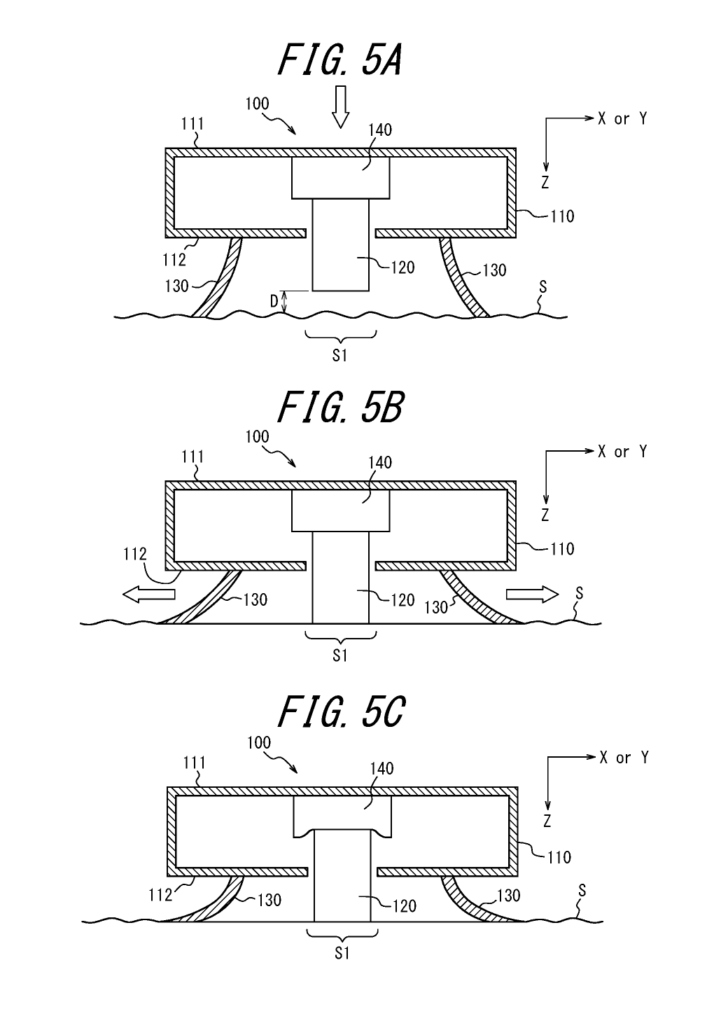

FIGS. 5A to 5C are lateral cross-sectional views illustrating a state in which the measuring apparatus 100 is pressed against the test site. FIGS. 5A to 5C are diagrams illustrating a state in which the measuring apparatus 100 illustrated in FIG. 4 is in contact with the test site of the subject to conduct the measurement. Reference numerals in FIGS. 5A to 5C are basically the same as those in FIG. 4.

FIG. 5A illustrates a state in which, at the start of the measurement by the measuring apparatus 100, a top of the protrusion 130 comes into contact with the skin around the test site of the subject. FIG. 5B is a diagram illustrating a state in which, for example, from the state in FIG. 5A the belt portions 114 and 115 are tightened, and thus the wearing portion 110 is pushed toward the test site of the subject. FIG. 5C is a diagram in which, for example, from the state in FIG. 5B the belt portions 114 and 115 are further tightened, and thus the wearing portion 110 is further pushed toward the test site of the subject.

FIG. 5A illustrates an example of a state in which, at the start of the measurement of the biological information by the measuring apparatus 100, the subject positions the sensor unit 120 on a subject's skin S of a test site S1. At this point, the top (a portion to come into contact with the subject's skin S) of the protrusion 130 is in light contact with the subject's skin S at a periphery of the test site S1. In this state, a detection surface of the sensor unit 120 is not in contact with the test site S1 and spaced apart therefrom by the distance D.

As illustrated in FIG. 5A, a surface of the subject's skin S has unevenness due to various factors such as, for example, skin wrinkles, keratin grooves on the skin surface, pores, sweat pores, and the like, and thus is normally in an unsmooth condition. When the detection surface of the sensor unit 120 is brought into close contact with the skin S in such a condition, the detection surface of the sensor unit 120 contacts with the test site S1 including, for example, the skin wrinkles or the keratin grooves on the skin surface. The skin wrinkles or the keratin grooves on the skin surface positioned at the detection surface of the sensor unit 120 becomes a factor for inhibiting accurate measurement of the biological information, thereby degrading the accuracy in the measurement by the measuring apparatus 100. Therefore, the measuring apparatus 100 according to the disclosure herein, in a state in which, by using the function of the protrusion 130, the skin of the test site S1 is stretched reducing an influence by the unevenness, the grooves, the wrinkles and the like, the detection surface of the sensor unit 120 is brought into close contact with the test site S1. For example, when the sensor unit 120 includes the light emitting element, the measuring apparatus 100 according to the disclosure herein may reduce scattering of the light from the skin wrinkles or the keratin grooves those are the factors to inhibit the accurate measurement of the biological information. As a result, the accuracy in the measurement by the measuring apparatus 100 is improved.

FIG. 5B is a diagram illustrating a state in which, from the state illustrated in FIG. 5A, the wearing portion 110 is pressed against the test site S1 in a direction of an arrow illustrated in FIG. 5A (in the positive direction of the Z-axis). When the wearing portion 110 is pressed against the test site S1, the protrusion 130 bends, reducing the distance D between the detection surface of the sensor unit 120 and the test site S1. FIG. 5B illustrates a state in which the distance D is zero, that is, the detection surface of the sensor unit 120 and the test site S1 are in contact with each other.

According to the disclosure herein, when the state illustrated in FIG. 5A shifts to the state illustrated in FIG. 5B, the protrusion 130 having the shape spreading toward the test site S1 further spreads in a direction indicated by an arrow illustrated in FIG. 5B (in a direction vertical to the Z-axis). Here, when the top of the protrusion 130 is in contact with the skin S at the periphery of the test site S1 and further spreads as the protrusion 130 bends, a part of the subject's skin S including the test site S1 is stretched. As illustrated in FIG. 5A, therefore, the part of the skin S originally having the unevenness including at least the test site S1 becomes smooth as illustrated in FIG. 5B.

FIG. 5C illustrates a state in which, from the state illustrated in FIG. 5B, the wearing portion 110 is further pressed against the test site S1 in the positive direction of the Z-axis. When the wearing portion 110 is further pressed against the test site S1, the protrusion 130 further spreads, whereby the detection surface of the sensor unit 120 is pressed against, and comes into close contact with, the test site S1. As described above, when the detection surface of the sensor unit 120 is pressed against the test site S1, the protrusion 130 functions to further stretch the part of the skin S including the test site S1. Since the distance D between the detection surface of the sensor unit 120 and the test site cannot become smaller than zero, further pressure applied after the state illustrated in FIG. 5B is absorbed by the elastic body 140 as illustrated in FIG. 5C. In this manner, the elastic body 140, in order to prevent the force to press the detection surface of the sensor unit 120 against the test site S1 from becoming excessively strong, also functions as a buffer material for allowing close contact with appropriate pressure.

In the disclosure herein, when the sensor unit 120 is not in contact with the test site of the subject (see FIG. 4), the protrusion 130 protrudes further than the sensor unit 120 toward of the test site. Also, when the sensor unit 120 comes into contact with the test site of the subject S1, the protrusion 130 contacts with periphery of the test site S1 and deforms further spreading toward the test site S1 (see FIGS. 5A to 5C). That is, when the wearing portion 110 is worn by the subject, the protrusion 130 contacts with the periphery of the test site S1 and deforms before the sensor unit 120 contacts with the test site S1, thereby stretching the skin of the test site S1 to be contacted by the sensor unit 120.

Here, the protrusion 130 may have various designs to deform in the spreading manner other than the design illustrated in FIGS. 5A to 5C. As described above, the protrusion 130 is typically formed to at least partially have elasticity or flexibility. Alternatively, for example, in the cross-sectional view of the protrusion 130 illustrated in FIGS. 5A to 5C, a thickness may be partially changed (for example, one portion alone of the cross-section is formed to be thin) such that the portion easily bends. A plurality of portions having a different thickness may be provided. In the cross-section of the protrusion 130 illustrated in FIGS. 5A to 5C, a plurality of materials with different hardness may be laminated so as to easily bend in a particular direction.

Also, the top (the portion to come into contact with the subject's skin S) of the protrusion 130 is a portion to stretch the skin of the test site S1 to be contacted by the sensor unit 120. Therefore, the top of the protrusion 130 may be designed to grip the subject's skin S without slipping thereon so as to be able to appropriately stretch the subject's skin S. A design to grip the skin S without slipping thereon may be substantialized by, for example, forming an uneven pattern at the top of the protrusion 130 or applying a coating agent such as silicone-acrylic resin and the like to the top of the protrusion 130.

As described above, when the detection surface of the sensor unit 120 is not in close contact with the test site S1 of the subject in an appropriate manner, the noise included in the measurement result tends to increase. When the detection surface of the sensor unit 120 is brought close contact into the test site S1 of the subject having the keratin grooves and the like on the subject's skin, simply pressing the sensor unit 120 against the test site S1 cannot achieve appropriate close contact. In the measuring apparatus 100 of the disclosure herein, the protrusion 130 is disposed around the sensor unit 120 and, when the sensor unit 120 contacts with the test site S1, stretches the test site S1. Thereby, the measuring apparatus 100 of the disclosure herein may bring the sensor unit 120 into close contact with the test site S1 while the protrusion 130 is stretching the keratin grooves and the like in the surface of the subject's skin. Accordingly, the measuring apparatus 100 of the disclosure herein may reduce the noise of the measurement and improve the accuracy in the measurement of the biological information.

Next, a variation of the disclosure herein will be described.

In the example illustrated in FIG. 3A, the protrusion 130 is described to surround the sensor unit 120. When the protrusion 130 has such a structure, forming the protrusion 130 with, for example, a light-shielding material and the like offers an advantage that, in the measurement of the biological information, the light from the light emitting element in the sensor unit 120 and the like is prevented from leaking. Also, there is another advantage that the photodetector in the sensor unit 120 is not affected by ambient light. In this manner, the protrusion 130 may be designed to prevent the ambient light from entering the sensor unit 120 when the wearing portion 110 is worn by the subject.

The protrusion 130 of the disclosure herein, from the viewpoint of stretching the skin of the test site S1 to be contacted by the sensor unit 120, does not necessarily need to be made of the light-shielding material in its entirety. For example, even when the protrusion 130 is at least partially made of the light-shielding resin, the function to stretch the skin of the test site S1 is not diminished at all.

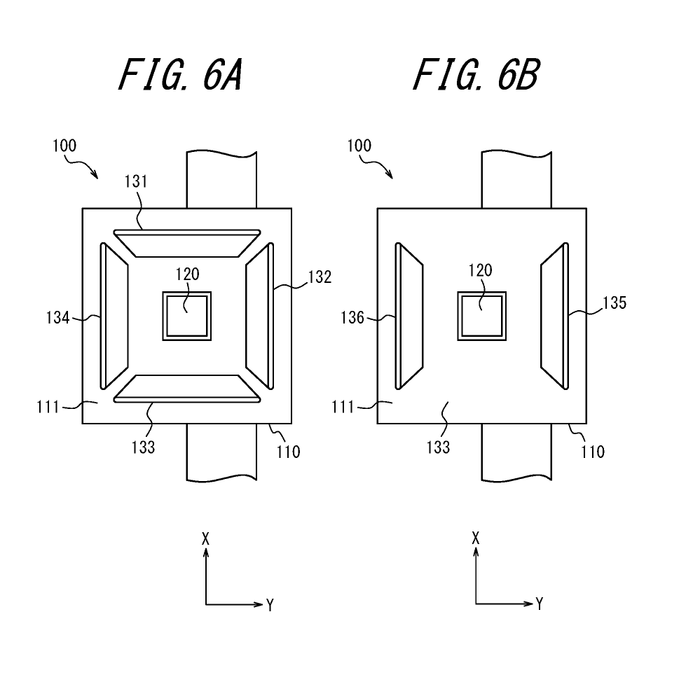

FIGS. 6A and 6B are elevation views illustrating the appearance of the measuring apparatus 100 in another example. In the measuring apparatus 100 of the disclosure herein, the structure of the protrusion 130 is not limited to that illustrated in FIGS. 3A and 3B but may have a structure illustrated in FIG. 6A and FIG. 6B, for example. Both FIG. 6A and FIG. 6B correspond to the elevation view of FIG. 3A illustrating the appearance of the measuring apparatus 100.

When the protrusion 130 is not required to have complete shielding function, the protrusion 130 does not need to be integrally formed as illustrated in FIG. 3A but, as illustrated in FIG. 6A, for example, may be formed with a plurality of members 131, 132, 133, and 134. In the example illustrated in FIG. 3A, in order for the protrusion 130 having the shape spreading toward the test site to deform and further spread, at least the top (the portion to come into contact with the subject's skin) of the protrusion 130 needs to be made of a stretching material. However, with the protrusions 131, 132, 133, and 134 as illustrated in FIG. 6A, when portions thereof joining the rear surface 111 of the wearing portion 110 are movable, the other portions thereof do not particularly need to have elasticity or flexibility. In this case, the portions other than the movable portions of the protrusion 130 may be made by using a material having a certain hardness such as a plastic, or a component for functioning as a core having a certain hardness may be embedded inside the material having elasticity or flexibility.

Also, although FIG. 6A illustrates the example in which the protrusion 130 is formed with a plurality of (four) members, the protrusion 130 may be, for example, integrally formed at least partially having a slit. That is, similarly to the example illustrated in FIG. 6A, slits may be formed to completely separate the protrusion 130 at four corners thereof. Alternatively, the portions of the protrusion 130 joining the rear surface 111 of the wearing portion 110 may be integrally formed, while the slit is formed from a middle of the protrusion 130 to the top (the portion to come into contact with the subject's skin) of the protrusion 130. Any number of such slits may be formed.

Further, from the viewpoint of stretching the skin of the test site S1 to be contacted by the sensor unit 120, the protrusion 130, as illustrated in FIG. 6B, for example, may have a design to stretch the skin of the test site S1 in at least one direction. In the example illustrated in FIG. 6B, when the wearing portion 110 is pressed against the test site S1 at the time of the measurement, the skin does not stretch in the X-axis direction but may stretch in the Y-axis direction alone.

According to the disclosure herein, as described above, the portion of the protrusion 130 protruding further than the sensor unit 120 toward the test site of the subject S1 contacts with a site other than the test site S1, thereby stretching the skin S of the test site S1. Also, the sensor unit 120, while being in contact with the skin S of the test site S1 being stretched as described above, acquires the biological information of the subject.

Next, a specific example of the measurement of a pulse wave velocity of the subject conducted by the measuring apparatus 100 will be described.

As described above, the measuring apparatus 100, when worn by the subject, measures the biological information of the subject. The biological information measured by the measuring apparatus 100 may be any biological information measurable by the sensor unit 120. Accordingly, the following is a description of a specific example of the measuring apparatus 100 of the disclosure herein which measures the pulse wave velocity by acquiring the pulse waves of the subject at two sites.

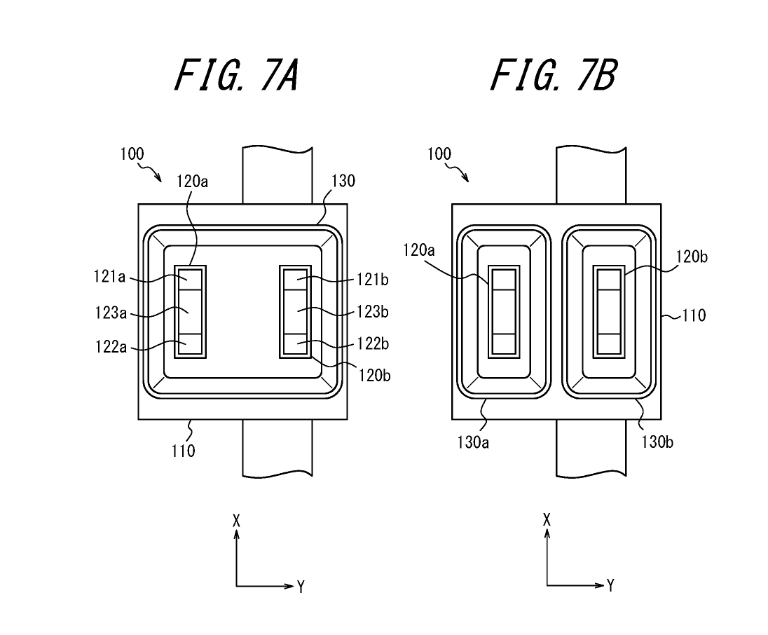

FIGS. 7A and 7B are diagrams illustrating an example in which the measuring apparatus 100 has two sensor units. FIGS. 7A and 7B, similarly to FIG. 3A and FIGS. 6A and 6B, are elevation views illustrating the appearance of the measuring apparatus 100.

As illustrated in FIGS. 7A and 7B, the measuring apparatus 100 of the present example has, as the sensor unit 120 described above, a first sensor unit 120a and a second sensor unit 120b. FIG. 7A illustrates an example in which the protrusion 130 is disposed around both the first sensor unit 120a and the second sensor unit 120b. FIG. 7B illustrates an example in which a protrusion 130a is disposed around the first sensor unit 120a, and a protrusion 130b is disposed around the second sensor unit 120b. Although the number of such sensor units 120 is not limited to two but may be any number depending on what is required for the measurement and specifications, the example having two sensor units 120 will be described here.

In the example illustrated in FIG. 7A, the protrusion 130, for the measurement, may stretch the skin of respective test sites to be contacted by the first sensor unit 120a and the second sensor unit 120b. In the example illustrated in FIG. 7A, further, the protrusion 130, for the measurement, may stretch the skin between the first sensor unit 120a and the second sensor unit 120b as well. On the other hand, in the example illustrated in FIG. 7B, the protrusion 130a and the protrusion 130b, for the measurement, cannot stretch the skin between the first sensor unit 120a and the second sensor unit 120b. However, in the example illustrated in FIG. 7B, the protrusion 130a and the protrusion 130b, for the measurement, may separately stretch the skin of the respective test sites to be contacted by the first sensor unit 120a and the second sensor unit 120b. According to the disclosure herein, as described above, the measuring apparatus 100 may have a plurality of sensor units 120, and the protrusion 130 may be disposed around the plurality of sensor units 120a and 120b.

The two sensor units 120a and 120b include a biosensor for acquiring the biological information of the subject. FIGS. 7A and 7B are diagrams illustrating examples of an arrangement of the biosensors of the sensor units 120a and 120b. Note that the wearing portion 110, the sensor unit 120, and the protrusion 130 are not limited to shapes thereof illustrated in FIGS. 7A and 7B but may have various shapes depending on what is required for the measurement and the specifications.

The sensor units 120a and 120b, while being in contact with the test sites of the subject, measure the biological information of the subject. In the present example, as illustrated in FIGS. 7A and 7B, the two biosensors, the first sensor unit 120a and the second sensor unit 120b, are arranged having a predetermined gap therebetween. The gap between the first sensor unit 120a and the second sensor unit 120b may be, for example, 10 to 30 mm The first sensor unit 120a and the second sensor unit 120b acquire pulse waves of different test sites by using an optical method. The pulse wave is a waveform acquired from the body surface representing a chronological change in a volume of the blood vessel caused by inflow of blood. In the present example, the plurality of sensor units 120a and 120b acquire the pulse wave serving as the biological information by using the optical method. In the disclosure herein, as described above, the biological information of the subject acquired by the sensor unit 120 may be the pulse wave.

As illustrated in FIG. 7A, the first sensor unit 120a includes, for example, two light emitting units 121a and 122a and a photodetector unit 123a. The second sensor unit 120b includes, for example, two light emitting units 121b and 122b and a photodetector unit 123b. FIG. 7B may have a similar configuration. A measuring beam is emitted to the test site from each of the light emitting units 121a, 122a, 121b, and 122b. By receiving a diffused light generated when the measuring beam reaches the photodetector units 123a and 123b after passing through the body, the pulse wave is acquired. The light emitting units 121a, 122a, 121b, and 122b include, for example, a light emitter such as, for example, LED (Light emitting diode), LE (Laser Diode), and the like. Also, the photodetector units 123a and 123b include a photodetector such as, for example, PD (Photodiode), PT (Phototransistor), and the like.

Note that, although in the above configuration each sensor unit 120 has two light emitting units and one photodetector unit, the present example may perform the measurement with a configuration in which each sensor unit 120 includes one light emitting unit and one photodetector unit. However, the following description assumes a configuration in which each sensor unit 120 includes two light emitting units and one photodetector unit.

The light emitting units 121a, 122a, 121b, and 122b emit any one of, for example, green light (a wavelength: 500 to 550 nm), red light (the wavelength: 630 to 780 nm), and near-infrared light (the wavelength 800 to 1600 nm). Since light of a long wavelength, as compared with light of a short wavelength, does not become attenuated before reaching a deeper portion of the body, the measurement of the biological information by using the light emitting element for emitting the near-infrared light is expected to improve measurement accuracy.

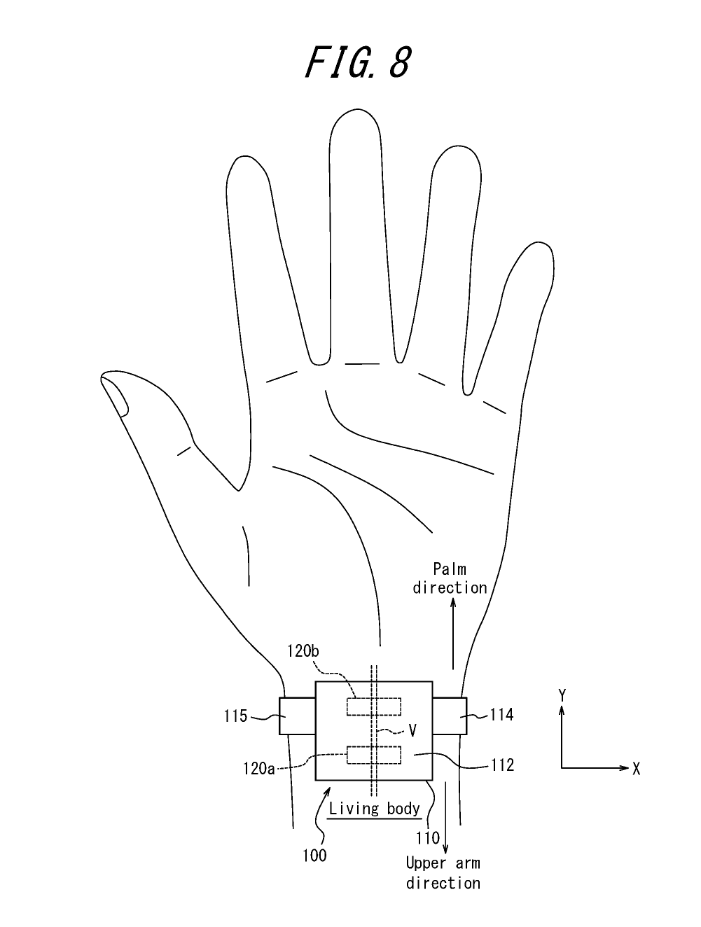

Referring to FIG. 8, next, an application method of the measuring apparatus 100 will be described.

FIG. 8 is a schematic diagram illustrating a state in which the measuring apparatus 100 illustrated in FIGS. 7A and 7B is worn by the subject. Similarly to the description with reference to FIG. 2, the subject winds the wearing portion 110 of the measuring apparatus 100 around the wrist for the measurement by the measuring apparatus 100. The subject, in winding the wearing portion 110 (or the belt portions 114 and 115 of the wearing portion 110) of the measuring apparatus 100 around the wrist, adjust positions of the sensor units 120a and 120b. At this time, the subject positions the wearing portion 110 such that the measuring beam is emitted from the light emitting units of the sensor units 120a and 120b to a predetermined blood vessel V such as the ulnar artery or the radial artery from which the biological information is acquired. Since the two sensor units 120a and 120b locate on the rear surface 111 of the wearing portion 110 rather than the front surface 112 thereof, the sensor units 120a and 120b are indicated by broken lines in FIG. 8.

As illustrated in FIG. 8, the measuring apparatus 100 is worn by the subject in such a manner that the two sensor units, the first sensor unit 120a and the second sensor units 120b, contact with the subject site such as, for example, the wrist. Especially, the first sensor unit 120a and the second sensor unit 120b may be adjusted to contact with the wrist by the subject himself in wearing the measuring apparatus in such a manner that the measuring beam is irradiated to the ulnar artery or the radial artery. At this time, the two sensor units 120a and 120b may be arranged along the predetermined blood vessel V of the subject in a state in which the wearing portion 110 is worn by the subject. In FIG. 8, the predetermined blood vessel V of the subject such as, for example, the ulnar artery or the radial artery is schematically illustrated by broken lines.

In the example illustrated in FIG. 8, also, as illustrated in FIG. 2, the wearing portion 110 (including the belt portions 114 and 115) may be a belt in the shape of an elongated strip. The measurement of the biological information is performed in a state in which, for example, the subject is wearing the wearing portion 110 of the measuring apparatus 100 around the wrist. In particular, the subject measures the biological information by wearing the wearing portion 110 around the wrist in such a manner that the plurality of sensor units 120a and 120b contact with the test site. The measuring apparatus 100, on the subject's wrist, measures the pulse wave velocity of the blood flowing in the ulnar artery or the radial artery.

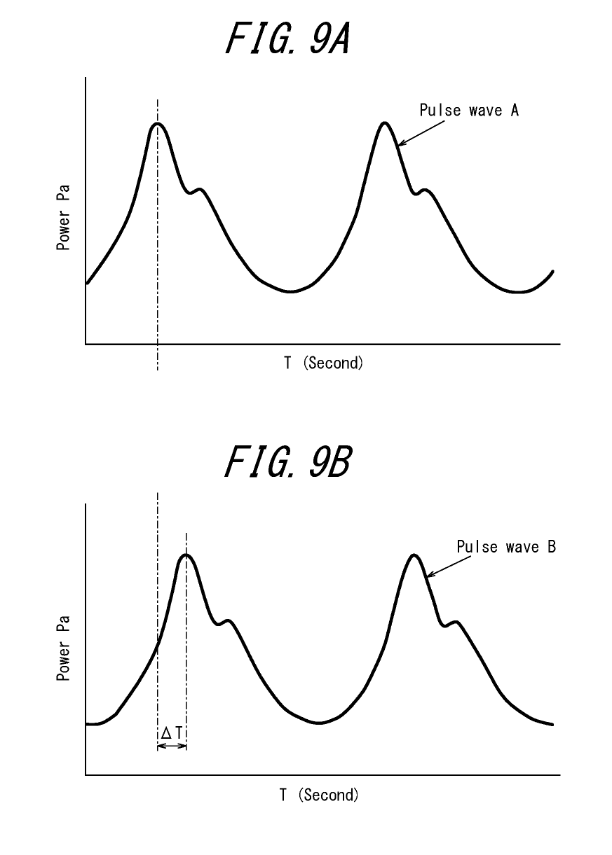

Referring to FIGS. 9A and 9B, next, based on two pulse waves acquired in the above configuration, a method of measuring the pulse wave velocity (Pulse Wave Velocity: PWV) between positions very close to each other on the wrist will be described.

FIGS. 9A and 9B are diagrams illustrating examples of the pulse wave acquired by the two biosensors. Note that, in the measuring apparatus 100, the first sensor unit 120a and the second sensor unit 120b are adjusted to locate over the radial artery of the subject, following the example illustrated in FIG. 8.

FIGS. 9A and 9B are diagrams vertically arranging and compares a pulse wave A acquired by the first sensor unit 120a in contact with a first test site A over the radial artery with a pulse wave B acquired by the second sensor unit 120b in contact with a second test site B over the radial artery. A power Pa indicated by a vertical axis in FIGS. 9A and 9B may represent, for example, the intensity of an output voltage from the photodetectors of the photodetector units of the first sensor unit 120a and the second sensor unit 120b. The two pulse waves acquired are synchronized in time. By using an interval ΔT (millisecond) of peak times of the two pulse waves acquired and an arrangement interval ΔD (mm) between the first sensor unit 120a and the second sensor unit 120b, the pulse wave velocity PWV (m/sec) may be calculated from the following formula:

PWV=ΔD/ΔT.

Next, the measurement of the pulse wave velocity conducted by the measuring apparatus 100 will be described from a functional point of view.

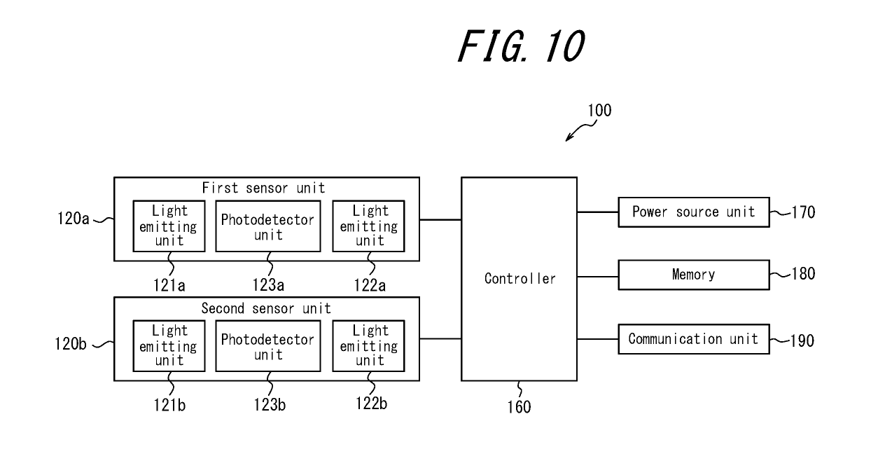

FIG. 10 is a functional block diagram illustrating a schematic configuration of the measuring apparatus 100 illustrated in FIG. 1. The measuring apparatus 100 includes the first sensor unit 120a, the second sensor unit 120b, a controller 160, a power source unit 170, a memory 180, and a communication unit 190. In the disclosure herein, all the first sensor unit 120a, the second sensor unit 120b, the controller 160, the power source unit 170, the memory 180, and the communication unit 190 may be disposed inside the wearing portion 110. Alternatively, in the disclosure herein, the controller 160, the power source unit 170, the memory 180, and the communication unit 190, other than the first sensor unit 120a and the second sensor unit 120b, may be disposed outside of the wearing portion 110 in an appropriate manner.

The first sensor unit 120a and the second sensor unit 120b include respective biosensors as described above and thus acquire the biological information from the test site. The first sensor unit 120a includes the light emitting units 121a and 122a and the photodetector unit 123a. The second sensor unit 120b includes the light emitting units 121b and 122b and the photodetector unit 123b.

The controller 160 is a processor for controlling and managing the measuring apparatus 100 in its entirety including each functional block thereof. Also, the controller 160 is a processor for calculating the pulse wave velocity based on the pulse wave acquired as the biological information. The controller 160 is constituted by using a processor such as CPU (Central Processing Unit) and the like for executing a program defining control procedure and a program for calculating the pulse wave velocity. Those programs may be stored in a storage medium such as, for example, the memory 180. In the disclosure herein, as described above, the measuring apparatus 100 may include the controller 160 for controlling the pulse wave velocity based on the pulse wave serving as the biological information of the subject acquired by the sensor unit 120.

The power source unit 170 includes, for example, a lithium ion battery and a control circuit for charging and discharging the battery and supplies power to the measuring apparatus 100 in its entirety, especially to the sensor unit 120.

The memory 180 may be constituted by using a semiconductor memory, a magnetic memory, or the like. The memory 180 stores various information and the programs for operating the measuring apparatus 100, as well as functioning as a work memory. The memory 180 may store, for example, a result of the measurement of the biological information acquired by the sensor unit 120.

The communication unit 190 performs a wired communication or a radio communication with an external apparatus, thereby transmitting and receiving various data. The communication unit 190, for example, communicates with the external apparatus storing the biological information of the subject and transmits, to the external apparatus, the result of the measurement of the biological information measured by the measuring apparatus 100.

Next, a measuring system including the measuring apparatus 100 according to the disclosure herein will be described.

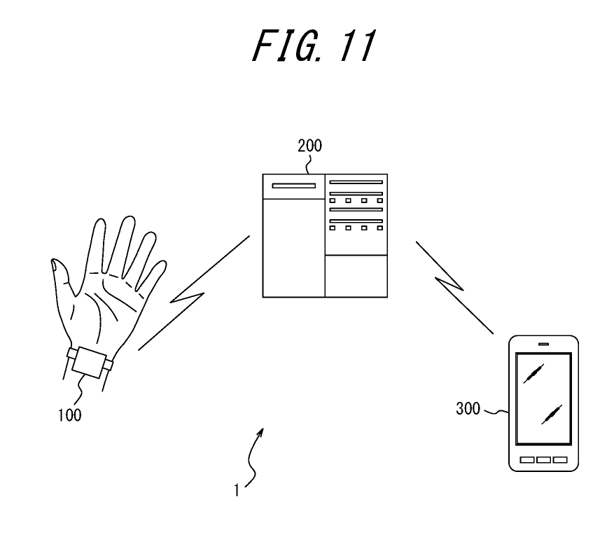

FIG. 11 is a diagram schematically illustrating the measuring system 1 including the measuring apparatus 100 according to the disclosure herein described above. The measuring system 1 may include, in addition to the measuring apparatus 100 according to the disclosure herein described above, a server 200 and a display unit 300.

The server 200 aggregates the biological information acquired by the measuring apparatus 100 and performs various information processing. For the aggregation of the biological information, the measuring apparatus 100, via a wired or wireless communication network, transmits the data to the server 200. The server 200 may be constituted by using a server apparatus having a general function capable of exchanging information with various terminals such as the measuring apparatus 100.

The display unit 300, based on the biological information acquired by the measuring apparatus 100, displays a result of the information processing performed by the server 200. That is, the display unit 300 displays information based on the biological information acquired by the measuring apparatus 100. The display unit 300 may be constituted by using a stand-alone display terminal such as, for example, an LCD, an organic EL display, and an inorganic EL display. Also, the display unit 300 may be constituted by using any terminal having such a display apparatus such as a smartphone, a tablet terminal, a laptop PC, a desktop PC, and the like.

In particular, the controller 160 of the measuring apparatus 100 transmits the biological information acquired by the measuring apparatus 100 from the communication unit 190 of the measuring apparatus 100 to the server 200. Upon reception of the biological information transmitted from the measuring apparatus 100, a controller built in the server 200 performs various information processing based on the biological information of the subject received. For example, the controller of the server 200 may store, in a memory of the server 200, the biological information acquired by the measuring apparatus 100 as chronological data associated with information about time at which the biological information is acquired.

Then, the controller of the server 200, for example, compares the data stored with past data of the same subject stored in the memory of the server 200 or data of another subject and, based on a result, may generate an optimal advice for the subject. Next, the communication unit of the server 200 may transmit information about the chronological data of the subject acquired and the advice generated to the display unit 300. Upon reception of the information transmitted as described above, the display unit 300 may display the data and the advice received. The measuring system 1 of the disclosure herein, as described above, includes the measuring apparatus 100 of the disclosure herein and the display unit 300 for displaying the information based on the biological information acquired by the measuring apparatus 100.

In the above measuring system 1, the measuring apparatus 100, the server 200, and the display unit 300 are assumed to be capable of performing respective control, although the measuring system 1 of the disclosure herein is not limited to such a configuration. For example, in the measuring system 1 of the disclosure herein, the measuring apparatus 100 or the display unit 300 may have a function unit having functions similar to those the memory and the controller built in the server 200. In this case, in the measuring system 1 the function of the server 200 does not necessarily need to intermediate, but the measuring apparatus 100 and the display unit 300 may directly exchange information therebetween.

It is to be understood that the disclosure herein is not limited to the above embodiment but may be modified or changed in various manners. For example, each function and the like included in each constituent and the like may be rearranged without logical inconsistency, so as to combine a plurality of constituents together or to separate them.

For example, in the above embodiment, the measuring apparatus 100 has one sensor unit 120 or two sensor units, first sensor unit 120a and the second sensor unit 120b. According to the disclosure herein, however, the number of a plurality of sensor units is not limited to one or two but may be any number of more than two. In this case, the configuration such as a shape of the wearing portion 110 and the like may be appropriately changed depending on the number of sensor units.

Also, the measuring apparatus 100 may include a notification unit for notifying the subject of the result of the measurement of the biological information. The notification unit may notify by employing any method that may be recognized by the subject. Here, the notification generated by the notification unit may be various notification including notification of start or end of predetermined measurement, notification of a result of the predetermined measurement, notification of predetermined warning, and the like. The notification unit may generate the notification by using a sound, an image, vibration, or a combination thereof. Note that the method employed by the notification unit is not limited to the above examples.

Also, although in the above embodiment the measuring apparatus 100 is used while being worn by the subject on the wrist, a using mode of the measuring apparatus 100 is not limited thereto. The measuring apparatus 100, depending on a location of the subject, may be used while being worn on the living body such as, for example, the ankle instead of the wrist. Also, although the disclosure herein describes the example in which the sensor unit 120 of the measuring apparatus 100 measures the biological information while being in contact with the test site of the subject, the disclosure herein is not limited thereto. When the protrusion 130 of the measuring apparatus 100 stretches the skin of the test site and reduces the influence by the unevenness, the grooves, the wrinkles and the like of the test site, the measuring apparatus 100 does not need the sensor unit 120 to contact with the test site of the subject but may reduce the noise in measurement.

Also, although in the above embodiment the measuring apparatus 100 is the apparatus for measuring the pulse wave velocity by way of example, the disclosure herein is not limited thereto. Since the measuring apparatus 100 may acquire the pulse wave highly accurately, the measuring apparatus 100 may be an apparatus for measuring the biological information based on the pulse wave. The measuring apparatus 100 may measure, for example, a blood pressure from the pulse wave acquired. The measuring apparatus 100 may measure, for example, a pulse from the pulse wave acquired. The biological information measured by the measuring apparatus 100 is not limited to the pulse wave. The measuring apparatus 100 may measure, for example, a blood flow. The measuring apparatus 100 may measure, for example, an amount of oxygen in blood or SpO2 (percutaneous oxygen saturation).

Also, although the above embodiment describes, by way of example, the measuring apparatus 100 as the apparatus for measuring the biological information by irradiating the light to the test site, the disclosure herein is not limited thereto. The measuring apparatus 100 may measure the biological information by, for example, irradiating ultrasonic waves to the test site.

1. A measuring apparatus comprising:
a wearing portion to be worn by a subject;
a sensor unit for acquiring a biological information of the subject while being supported by the wearing portion and in contact with a test site of the subject; and
a protrusion disposed around the sensor unit, wherein
the protrusion protrudes further than the sensor unit toward the test site when the sensor unit is not in contact with the test site, and
the protrusion, when the wearing portion is worn by the subject, contacts with a periphery of the test site and deforms before the sensor unit contacts with the test site, thereby stretching the skin of the test site to be contacted by the sensor unit.

2. The measuring apparatus according to claim 1, wherein the protrusion prevents ambient light from entering the sensor unit when the wearing portion is worn by the subject.

3. The measuring apparatus according to claim 1, wherein the protrusion is at least partially made of a light-shielding resin.

4. The measuring apparatus according to claim 1, wherein a portion of the sensor unit to come into contact with the test site includes a convex shape.

5. The measuring apparatus according to claim 1, comprising a plurality of sensor units and the protrusion disposed around the plurality of sensor units.

6. The measuring apparatus according to claim 5, wherein the plurality of sensor units are arranged run along a predetermined blood vessel of the subject when the wearing portion is worn by the subject.

7. The measuring apparatus according to claim 1, wherein the wearing portion is a belt to be worn by the subject on the wrist.

8. The measuring apparatus according to claim 1, wherein the biological information is a pulse wave.

9. The measuring apparatus according to claim 8, comprising a controller for calculating a pulse wave velocity based on the pulse wave.

10. A measuring apparatus comprising:
a sensor unit for acquiring biological information of a test site; and
a protrusion disposed around the sensor unit, wherein
the protrusion, when a portion thereof protruding from the sensor unit toward the test site contacts with a part other than the test site, stretches the skin of the test site, and
the sensor unit acquires the biological information while being in contact with the skin of the test site being stretched.

11. A measuring apparatus comprising:
a wearing portion to be worn by a subject;
a sensor unit for acquiring biological information of the subject while being supported by the wearing portion and in contact with a test site of the subject; and
a protrusion disposed around the sensor unit, wherein
the protrusion at least partially has a shape spreading toward the test site and, when the sensor unit is not in contact with the test site, protrudes further than the sensor unit toward the test site, and
the protrusion, when the sensor unit contacts with the test site, contacts with a periphery of the test site and deforms further spreading the shape.

12. The measuring apparatus according to claim 11, wherein the protrusion at least partially has elasticity or flexibility.

13. The measuring apparatus according to claim 11, wherein the protrusion at least partially has a slit.

14. A measuring system comprising:
the measuring apparatus according to claim 1; and
a display unit for displaying information based on biological information acquired by the measuring apparatus.
